(19) United States
(12) Patent Application Publication
(10) Pub. No.: US 2021/0244689 A1
(43) Pub. Date: Aug. 12, 2021

(54) METHOD OF TREATING POSTPRANDIAL INFLAMMATION

(71) Applicant: DASMAN DIABETES INSTITUTE, DASMAN (KW)

(72) Inventors: FATEMA AL-RASHED, DASMAN (KW); RASHEED AHMAD, DASMAN (KW)

(21) Appl. No.: 16/783,775

(22) Filed: Feb. 6, 2020

(51) Int. Cl. A61K 31/15; A61K 9/00; A61P 29/00

(52) U.S. Cl. CPC A61K 31/15; A61P 29/00; A61K 9/0095

(57) ABSTRACT

A method of treating postprandial inflammation may include administering to a subject an edible composition comprising the ACSL1 inhibitor within hours of the subject consuming a food. The edible composition may be administered by consumption by the subject at the same time as the subject consumes the food. The food may be high in saturated fatty acids.

### BACKGROUND

### 1. Field

The disclosure of the present patent application relates to formulations and methods useful in treating inflammation, and particularly to formulations and methods of reducing or regulating an inflammatory response due to diet based on a long chain acyl-CoA synthetase (ACSL) inhibitor.

### 2. Description of the Related Art

Food items in contemporary diets are often high in saturated fatty acids (SFA), such as palmitic acid. SFA are known to up-regulate inflammation upon consumption. Acute inflammatory responses can be initiated by food consumption, particularly high fat food consumption, such responses being called postprandial inflammation. Postprandial inflammation arises primarily from lipaemia caused by increased chylomicron formation and triacylglyceride (TAG) content in circulation following food consumption. Consuming a meal high in SFA may cause particularly acute postprandial inflammatory effects, which could further cause or aggravate metabolic conditions, such as insulin resistance or diabetes, or existing inflammatory conditions, such as arthritis. In lieu of dietary intervention, a pharmaceutical intervention is desired to prevent or minimize postprandial inflammation.

Acyl CoA synthetases (ACSL), particularly ACSL1, are major enzymes responsible for converting free fatty acids taken in through diet into several lipid subclasses usable as energy sources, cellular building blocks or cellular communication means. Free fatty acids, such as SFA, can only be utilized by the body after their activation or catalyzation by Acyl-CoA. In particular, work by the present inventors has shown that Acyl-CoA activation is important for tumor necrosis factor alpha (TNF-α) signaling, TNF-αbeing a critical pro-inflammatory cytokine. The use of ACSL inhibitors was also shown to stop TNF-αsignaling, thus stopping inflammatory responses in vitro (Al-Rashed, F., Ahmad, Z., Iskandar, M. A., Tuomilehto, J., Al-Mulla, F., & Ahmad, R. (Mar. 8, 2019). TNF-α induces a pro-inflammatory phenotypic shift in monocytes through ACSL1: relevance to metabolic inflammation. Cell Physiol Biochem, 52(3), 397-407). There are a limited number of TNF-α inhibitors available, and most become unusable because of tolerance within a year.

While dietary fatty acids are essential for several metabolical processes, certain saturated fatty acids, such as palmitic acid (PA), induce macrophage foaming and pro-inflammatory responses. These damaging processes might be limited in normal weight individuals, but in obese subjects, the increased amount of adipose tissue acts as a constant fuel, triggering a sustained or extreme monocyte/macrophage pro-inflammatory response. This also triggers macrophages to infiltrate adipose tissue, producing obesity-related inflammation. A means of blocking bad fatty acids during high intake of free fatty acids would break this cycle and prevent further pro-inflammatory response and damage.

Thus, a formulation and method of treating or preventing postprandial inflammation is desired.

### SUMMARY

A formulation for treating inflammation according to the present disclosure includes an edible composition comprising an ACSL1 inhibitor or a pharmaceutically acceptable salt thereof. The edible composition may be a beverage or soluble additive to a beverage, and the beverage may be a carbonated beverage.

In one embodiment, the present subject matter relates to a method of treating postprandial inflammation, or inhibiting a postprandial inflammatory response, in a subject, comprising administering to a subject in need thereof an edible composition comprising an ACSL1 inhibitor or a pharmaceutically acceptable salt thereof in an amount effective to treat the postprandial inflammation or inhibit the postprandial inflammatory response. In an embodiment, the composition is administered within hours of the subject consuming a food. In an embodiment, the edible composition is administered by consumption by the subject and the subject consumes the edible composition at about the same time as the subject consumes the food. The food may be high in SFA.

The composition may act as a neutralizer to acute effects of fatty acids ingested by the subject during consumption of food, such as fast food, rich in fatty acids, such as SFA, thereby preventing or minimizing the acute effects of said SFA.

These and other features of the present subject matter will become readily apparent upon further review of the following specification.

### BRIEF DESCRIPTION OF THE DRAWINGS

FIG. 1 shows a schematic of the ACSL pathway and the place of action of an ACSL inhibitor.

FIGS. 2A and 2B show the results of studies demonstrating the effects of an exemplary ACSL-1 inhibitor (Triacsin C) treatment on lipid accumulation in macrophage cells in response to an inflammation cytokine. FIG. 2A shows cell culture lipid droplet accumulation (red) in cells in culture; FIG. 2B shows the lipid accumulation in cells determined by flow cytometry (FACS) analysis.

FIGS. 3A, 3B and 3C show protein or gene expression levels for inflammation or infiltration markers IL-B protein, TNF-α mRNA and CD11b mRNA, respectively, for macrophage cells treated with the exemplary ACSL-1 inhibitor.

Similar reference characters denote corresponding features consistently throughout the attached drawings.

### DETAILED DESCRIPTION OF THE PREFERRED EMBODIMENTS

The formulation for treating postprandial inflammation according to the present disclosure includes a composition comprising an ACSL1 inhibitor or a pharmaceutically acceptable salt thereof. In an embodiment, the composition is suitable for oral administration to a subject. The composition may be edible, such as a beverage or soluble additive to a beverage, and the beverage may be a carbonated beverage. In an embodiment, the ACSL1 inhibitor is a known inhibitor, such as Triacsin C, 2-Fluoropalmitic acid or Adenosine 5′-hexadecylphosphate (AMPC16).

In another embodiment, the present subject matter relates to a method of treating inflammation comprises administering to a subject in need thereof a composition comprising the ACSL1 inhibitor or a pharmaceutically acceptable salt thereof within a time frame of hours of the subject consuming a food. The time frame may be before, during or after consuming the food. In an embodiment, the composition is administered by consumption by the subject at about the same time as the subject consumes the food, namely immediately before, during or immediately after the subject consumes the food. The food may be high in SFA. The subject may be afflicted with any condition characterized with elevated TNF-α. Such conditions may include, without limitation, obesity, diabetes and arthritis. The subject may be an obese person, or otherwise a person with excessive adipose tissue.

The present composition can act as a neutralizer to acute effects of fatty acids ingested by the subject during consumption of foods, such as fast foods, rich in fatty acids such as SFA, thereby preventing or minimizing the acute effects of said SFA. After consumption, fatty acids become emulsified in the duodenum, where each three fatty acids get packaged with a glycerol group to produce “triglycerides”. These triglycerides circulate throughout the body via apolipoproteins to distribute energy and essential fatty acids needed for other processes. An edible form of an ACSL inhibitor can prevent the formation of triglycerides rich in long chain fatty acids, such as palmitic acid (PA). Alternatively, a direct injection of an ACSL inhibitor into inflamed adipose tissue could also be used to reduce inflammation at the site. ACSL-1 inhibitors useful herein may be any compound that inhibits ACSL-1, such as, by way of non-limiting examples, Triacsin C, 2-Fluoropalmitic acid or AMPC16.

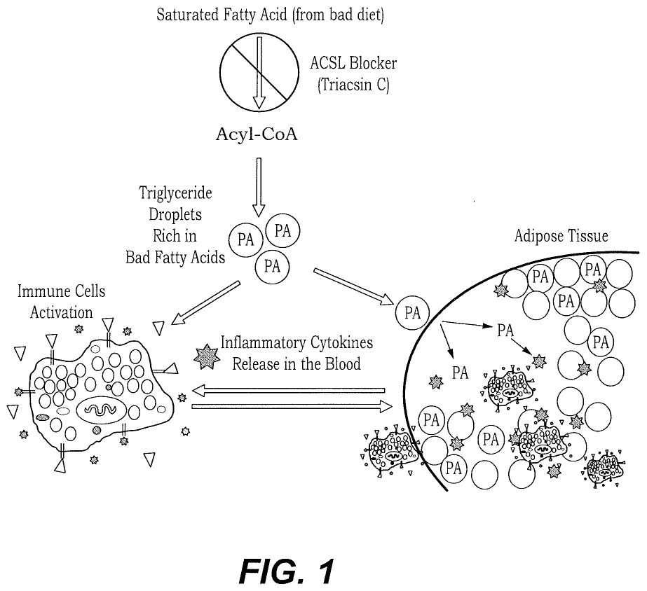

FIG. 1 shows a proposed schematic of the ACSL target pathway. Free fatty acids can only be utilized by the body after their activation to become Acyl-CoA. This process is catalyzed by Acyl-CoA synthetase (ACSL). Even though dietary fatty acids are essential for several metabolic processes, saturated fatty acids such as palmitic acid (PA) induce potentially harmful, pathogenic, or otherwise unwanted responses, such as macrophage foaming and pro-inflammatory responses. Blocking bad fatty acid activation through administration of an ACSL inhibitor shortly before, during or shortly after high intake of free fatty acids can stop the accumulation of triglyceride droplets in the blood, which could trigger an immune response, thereby potentially preventing further pro-inflammatory response and damage. Additionally, Acyl-CoA activation is important for TNF-α signaling, and the use of ACSL inhibitors can stop TNF-α signal thus stopping inflammatory responses. Therefore, any condition with elevated TNF-α should benefit from such an inhibitor. In particular, a postprandial acute inflammatory response should benefit from such an inhibitor, specifically administered soon before, at the same time as, or soon after eating.

Salts encompassed within the term “pharmaceutically acceptable salts” as used herein refer to non-toxic salts of the ACSL-1 inhibitors which are generally prepared by reacting a free base with a suitable organic or inorganic acid or by reacting an acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid. As examples of pharmaceutically acceptable salts with bases may be mentioned the lithium, sodium, potassium, calcium, aluminum, magnesium, titanium, ammonium, meglumine or guanidinium salts.

As used herein, an “effective” amount refers to a therapeutically effective amount for the prevention and/or inhibition of postprandial increase in inflammatory markers or monocyte polarization to pro-inflammatory macrophages. Inflammatory markers may include, for example, C-reactive proteins (CRP); cytokines, such as TNF-α, interleukins, such as IL-6; or adhesion molecules, such as VCAM or ICAM, GDF-15 or ST2.

As used herein, the term “subject” may refer to a mammal such as a human.

As used herein, the term “about” when used to modify a numeral, means within 10% of the numeral's value.

In particular, ACSL-1 inhibitors are shown in the following examples to prevent monocyte polarization to pro-inflammatory macrophages.

### EXAMPLE 1

### Materials and Methodologies

Cell culture: Human monocytic leukemia cell line (THP-1) cells were purchased from American Type Culture Collection (ATCC). Cell cultures were maintained in RPMI-1640 culture medium (Gibco, Life Technologies, Grand Island, USA) supplemented with 10% fetal bovine serum (Gibco, Life Technologies, Grand Island, N.Y., USA), 2 mM glutamine (Gibco, Invitrogen, Grand Island, N.Y., USA), 1 mM sodium pyruvate, 10 mM HEPES, 100 ug/ml Normocin 50 U/ml penicillin and 50 μg/ml streptomycin (P/S; Gibco, Invitrogen, Grand Island, N.Y., USA) and incubated at 37° C. (with humidity) in 5% CO2.

Cell stimulation: Prior to stimulation, THP-1 cells were plated in 24-well plates (Costar, Corning Incorporated, Corning, N.Y., USA) at 5×105 cells/well cell density unless indicated otherwise. Cells were incubated with either Triacsin C, a Long chain acyl-CoA synthetase (ACSL-1) inhibitor or Etomoxir, a carnitine palmitoyltransferase-1 (CPT-1) inhibitor (CPT-1 is a mitochondrial enzyme involved in fatty acid (3-oxidation). Etomoxir is an irreversible inhibitor of carnitine palmitoyltransferase-1 (CPT-1), and is used widely as a small-molecule inhibitor of fatty acid oxidation (FAO). FAO is the process by which active fatty acids stored in triglycerides are released when energy is needed. Blocking FAO stops fatty acids from being liberated and released from adipose cells. Etomoxir acts as a positive control, in that it acts oppositely from ACSL. ACSL inhibitors prevents fat from going into the cells (fatty acid depletion), while Etomoxir prevent fat from being burned (fatty acid accumulation). A control of no treatment, also referred to as vehicle, was also used in the experiments.

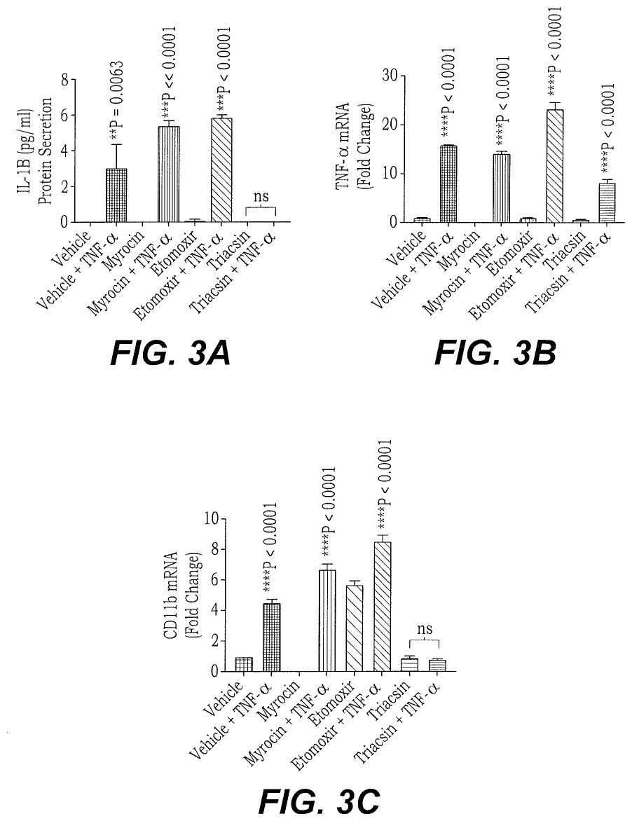

All lipid inhibitors were purchased from Invivogen (InvivoGen, San Diego, Calif., USA). Cultures were then stimulated with TNF-α (10 ng/ml) overnight at 37° C. Cells were harvested for cell sorting (FACS) analysis, RNA isolation for gene expression and conditioned media were collected for measuring IL-1β secretion levels. IL-1β is a pro-inflammatory cytokine indicative of inflammation by secretion level (see ELISA results in FIG. 3A). Exemplary RNA gene expression results are shown for, e.g., TNF-α (FIG. 3B) and other monocyte inflammatory and infiltration markers, such as CD11b (FIG. 3C) gene regulators. Conditioned media were collected and stored at −80° C.

Macrophage transformation: Prior to macrophage transformation of THP-1 monocytes, cells were plated at 106 cells per ml in 24 well plates (unless specified otherwise) and pre-treated with different lipid metabolite inhibitors (Triacsin C or Etomoxir, as above) or media alone as the vehicle condition for 1 hour at 37° C. The cells being treated as above were then further treated with 10 ng/ml phorbol 12-myristate 13-acetate (PMA, Sigma) for 24 hours at 37° C. to facilitate differentiation into macrophages. The monocyte-derived macrophages were washed three times with phosphate-buffered saline (PBS) and further incubated for 48 h in serum-free RPMI (37° C., 5% CO2). Resulting cultures were subject to the assays mentioned below and data were collected for the different analysis.

Extracellular staining flow cytometry: After experimental or control treatments, the macrophages were lifted by using phosphate-buffered saline (PBS) supplemented with ethylenediaminetetraacetic acid (EDTA). Collected cells were then centrifuged and washed 3 times. The monocytic cells (1×10⁶ cells/mL) were resuspended in FACS staining buffer (BD Biosciences) and blocked with human IgG (Sigma; 20 μg) for 30 minutes on ice. Cells were washed three times with FACS buffer and resuspended in 2% paraformaldehyde. Cells were centrifuged and resuspended in FACS buffer for FACS analysis (FACSCanto II; BD Bioscience, San Jose, USA). FACS data analysis was performed using BD FACSDiva™ Software 8 (BD Biosciences, San Jose, USA). Cells were sorted according to their size and granulation using the protocol presented by Lee et al., 2004 for lipid measurement (Lee, Y. H., Chen, S. Y., Wiesner, R. J., & Huang, Y. F. (2004). Simple flow cytometric method used to assess lipid accumulation in fat cells. Journal of lipid research, 45(6), 1162-1167), the content of which is incorporated herein.

Oil Red O staining: To determine the degree of lipid accumulation, the accumulation of cytoplasmic triglycerides in cells were measured by staining with Oil Red O (SigmaAldrich, St Louis, Mo., USA). Briefly, the cells were washed three times in PBS, then fixed with 4% paraformaldehyde for 30 min at room temperature. The cells were incubated with 60% isopropanol for 5 min, then washed with deionized water. The cells were incubated with 0.5% Oil Red O solution for 5 min to label lipid droplets. After staining, the cells were washed several times with deionized water to remove excess stain, then counterstained with hemotoxylin to visualize general structure. The stained cells were photographed at x40 magnification using a Zeiss Axio Inverted Microscope.

### EXAMPLE 2

### Results

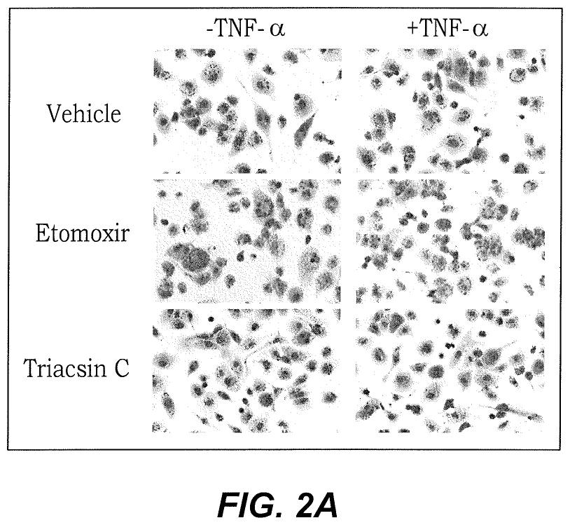

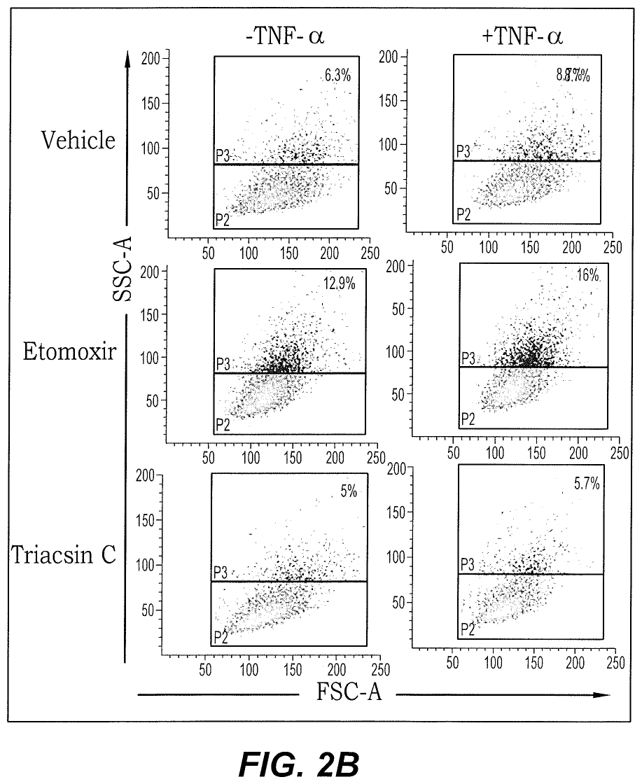

Results of the Oil red O staining show Triacsin C prevents lipid accumulation in response to TNF-α, particularly relative to positive controls of vehicle and Etomoxir treatments, alone (FIG. 2A). These results confirm the findings indicated in the FACS analysis shown in FIG. 2B. Treated cultured cells were visualized under a microscope. Red color in FIG. 2A represents fat/lipid accumulation within the macrophages.

FIG. 2B shows dot plots of the results from the previously described FACS analysis. Both the size of the cells and their granulation are used to assess lipid accumulation. Green dots represent the general population size and blue dots represent those that accumulated fat. Both vehicle treated and Etomoxir treated cells showed increased lipid accumulation in response to the presence of TNF-α (percentages in top right of plots). In contrast, the exemplary ASCL-1 inhibitor (Triacsin C) treated cells exhibited no significant change in apparent lipid accumulation.

FIG. 3A shows ELISA measurements of protein expression levels of pro inflammatory cytokine IL-1B and FIGS. 3B and 3C show gene expression (mRNA) levels of TNF-α and another inflammation marker CD11b, respectively, in macrophage cells treated as above. Only Triacsin C treated cells showed little to no change in inflammatory markers in response to TNF-α treatment. Thus, treatment with Triacsin C not only reduces or prevents accumulation of lipids in macrophages, but reduces or prevents a subsequent inflammatory response.

It is to be understood that the formulation and method of treating postprandial inflammation is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and us.

1. A method for inhibiting an inflammatory response in a subject, comprising administering an ACSL-1 inhibitor selected from the group consisting of Triacsin C, 2-Fluoropalmitic acid, or Adenosine 5′-hexadecylphosphate, or a pharmaceutically acceptable salt thereof to a subject in need thereof, wherein the ACSL-1 inhibitor is formulated as an edible composition in an amount effective to inhibit an increase in inflammatory markers or monocyte polarization to pro-inflammatory macrophages resulting from the subject's consumption of a food that is high in at least one saturated fatty acid; and wherein the ACSL-1 inhibitor is administered within three hours before the subject's consumption of the food.

2. (canceled)

3. The method of claim 1, wherein the ACSL-1 inhibitor is Triacsin C.

4. The method of claim 3 wherein the subject has one or more of obesity, insulin resistance, diabetes and arthritis.

5. The method of claim 4, wherein the subject is obese.

6. (canceled)

7. The method of claim 1, wherein the ACSL-1 inhibitor is administered in a beverage.

8. The method of claim 7, wherein the ACSL-1 inhibitor is administered in a carbonated beverage.

9. (canceled)

10. The method of claim 1, wherein the ACSL-1 inhibitor is administered at about the same time as the food provoking the postprandial inflammatory response.

11. (canceled)

12. The method of claim 1, wherein the at least one saturated fatty acid is palmitic acid.

13. (canceled)
